(19) United States
(12) Patent Application Publication
(10) Pub. No.: US 2009/0030372 A1
(43) Pub. Date: Jan. 29, 2009

(54) Liquid/gas separator for surgical cassette

(76) Inventor: Filip Finodeyev, Laguna Niguel, CA (US)

Correspondence Address: ALCON, IP LEGAL, TB4-8, 6201 SOUTH FREEWAY, FORT WORTH, TX, 76134, US

(21) Appl. No.: 12/119,702

(22) Filed: May 13, 2008

Related U.S. Application Data
(60) Provisional application No. 60/951,824, filed on Jul. 25, 2007.

(51) Int. Cl. A61M 1/00

(52) U.S. Cl. 604/122

(57) ABSTRACT

A surgical cassette having an aspiration chamber and an aspiration source chamber. The aspiration chamber has a liquid/gas separating structure. The liquid/gas separating structure prevents bubble and liquid ingress into the aspiration source chamber and facilitates accurate, reliable measurement of the fluid level in the aspiration chamber.

This application claims the priority of U.S. Provisional Application No. 60/951,824 filed Jul. 25, 2007.

### FIELD OF THE INVENTION

The present invention generally pertains to a surgical cassette for use with microsurgical systems, and more particularly to such cassettes for use with ophthalmic microsurgical systems.

### DESCRIPTION OF THE RELATED ART

During small incision surgery, and particularly during ophthalmic surgery, small probes are inserted into the operative site to cut, remove, or otherwise manipulate tissue. During these surgical procedures, fluid is typically infused into the eye, and the infusion fluid and tissue are aspirated from the surgical site. The types of aspiration systems used are generally characterized as either flow controlled or vacuum controlled, depending upon the type of pump used in the system. Each type of system has certain advantages.

Vacuum controlled aspiration systems are operated by setting a desired vacuum level, which the system seeks to maintain. Flow rate is dependent on intraocular pressure, vacuum level, and resistance to flow in the fluid path. Actual flow rate information is unavailable. Vacuum controlled aspiration systems typically use a venturi or diaphragm pump. Vacuum controlled aspiration systems offer the advantages of quick response times, control of decreasing vacuum levels, and good fluidic performance while aspirating air, such as during an air/fluid exchange procedure. Disadvantages of such systems are the lack of flow information resulting in transient high flows during phacoemulsification or fragmentation coupled with a lack of occlusion detection. Vacuum controlled systems are difficult to operate in a flow controlled mode because of the problems of non-invasively measuring flow in real time.

Flow controlled aspiration systems are operated by setting a desired aspiration flow rate for the system to maintain. Flow controlled aspiration systems typically use a peristaltic, scroll, or vane pump. Flow controlled aspiration systems offer the advantages of stable flow rates and automatically increasing vacuum levels under occlusion. Disadvantages of such systems are relatively slow response times, undesired occlusion break responses when large compliant components are used, and vacuum can not be linearly decreased during tip occlusion. Flow controlled systems are difficult to operate in a vacuum controlled mode because time delays in measuring vacuum can cause instability in the control loop, reducing dynamic performance.

One currently available ophthalmic surgical system, the MILLENIUM system from Storz Instrument Company, contains both a vacuum controlled aspiration system (using a venturi pump) and a separate flow controlled aspiration system (using a scroll pump). The two pumps can not be used simultaneously, and each pump requires separate aspiration tubing and cassette.

Another currently available ophthalmic surgical system, the ACCURUS® system from Alcon Laboratories, Inc., contains both a venturi pump and a peristaltic pump that operate in series. The venturi pump aspirates material from the surgical site to a small collection chamber. The peristaltic pump pumps the aspirate from the small collection chamber to a larger collection bag. The peristaltic pump does not provide aspiration vacuum to the surgical site. Thus, the system operates as a vacuum controlled system.

In both vacuum controlled aspiration systems and flow controlled aspiration systems, the liquid infusion fluid and ophthalmic tissue aspirated from the surgical site are directed into an aspiration chamber within a surgical cassette. This results in bubbles forming in the aspiration chamber which often cause difficulties in obtaining an accurate measurement of the fluid level in the aspiration chamber. In vacuum controlled aspiration systems, the aspiration chamber in the surgical cassette is fluidly coupled to a source of vacuum within a surgical console. Any bubbles present in the aspiration chamber may travel to the source of vacuum, resulting in liquid ingress into the surgical console and an increased potential for biocontamination and corrosion of internal components. Therefore, it is important to protect the source of vacuum from liquid, while maintaining the ability to aspirate air from above the partially liquid-filled aspiration chamber. In the past, hydrophobic filter media were incorporated into the fluid line between the vacuum source and aspiration chamber to provide such protection. However, such filter media delayed air flow and correspondingly increased the fluidic response time of the surgical system. In addition, large air chambers or long fluid paths have been incorporated into conventional ophthalmic surgical systems to reduce the likelihood of liquid reaching the source of vacuum. However, such added volumes of air increased the fluidic response time of the surgical system due to an increased amount of compressible fluid in the system.

Accordingly, a need continues to exist for an improved method of protecting a source of vacuum in the aspiration system of a microsurgical system from liquid and obtaining an accurate measurement of the fluid level within the aspiration chamber of a surgical cassette.

### SUMMARY OF THE INVENTION

The present invention relates to a surgical cassette having an aspiration source chamber and an aspiration chamber disposed therein. The aspiration chamber includes an overflow chamber, a sensing chamber, and a liquid/gas separating structure dividing the sensing chamber into an anterior section and a posterior section. The separating structure includes a converging nozzle fluidly coupled to the anterior section, a curved deflector disposed in the overflow chamber, and a drain channel disposed in the sensing chamber and fluidly coupled to the overflow chamber and the anterior section. The aspiration chamber further includes a first opening to the anterior section for receiving a liquid/gas mixture from a surgical device, an exit from the converging nozzle for directing the liquid/gas mixture toward a concave surface of the deflector, and a second opening disposed outside a convex surface of the deflector and fluidly coupling the overflow chamber and the aspiration source chamber.

### BRIEF DESCRIPTION OF THE DRAWINGS

For a more complete understanding of the present invention, and for further objects and advantages thereof, reference is made to the following description taken in conjunction with the accompanying drawings, in which:

FIG. 1 is a schematic diagram illustrating aspiration control in a microsurgical system including a surgical cassette; and

FIG. 2 is an enlarged, front, sectional, schematic view of an aspiration chamber and an aspiration source chamber of the surgical cassette of FIG. 1 having a liquid/gas separating structure according to a preferred embodiment of the present invention.

### DETAILED DESCRIPTION OF THE PREFERRED EMBODIMENTS

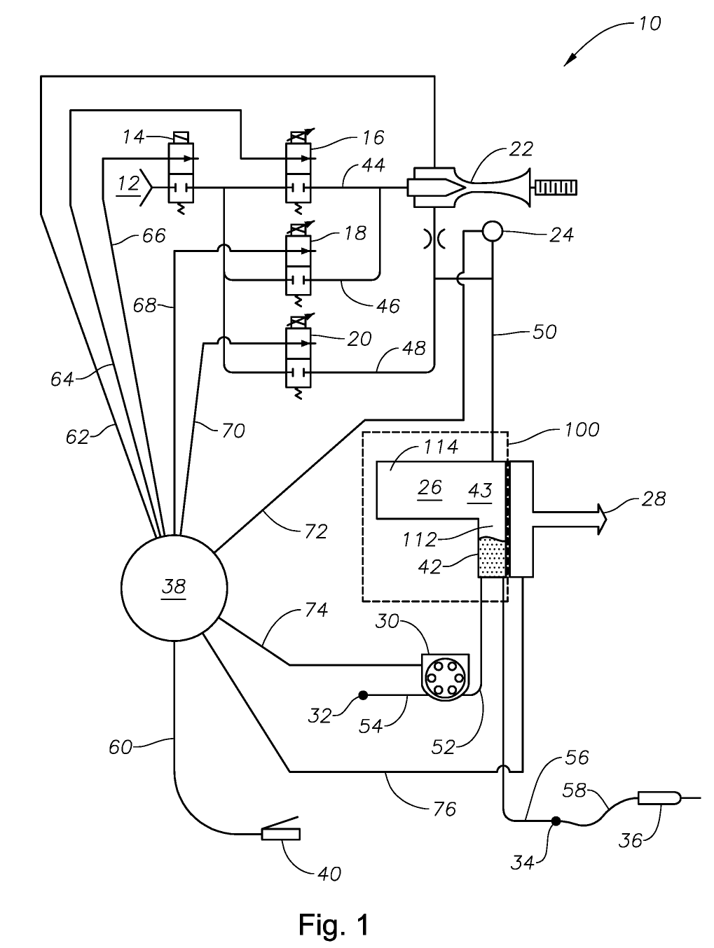

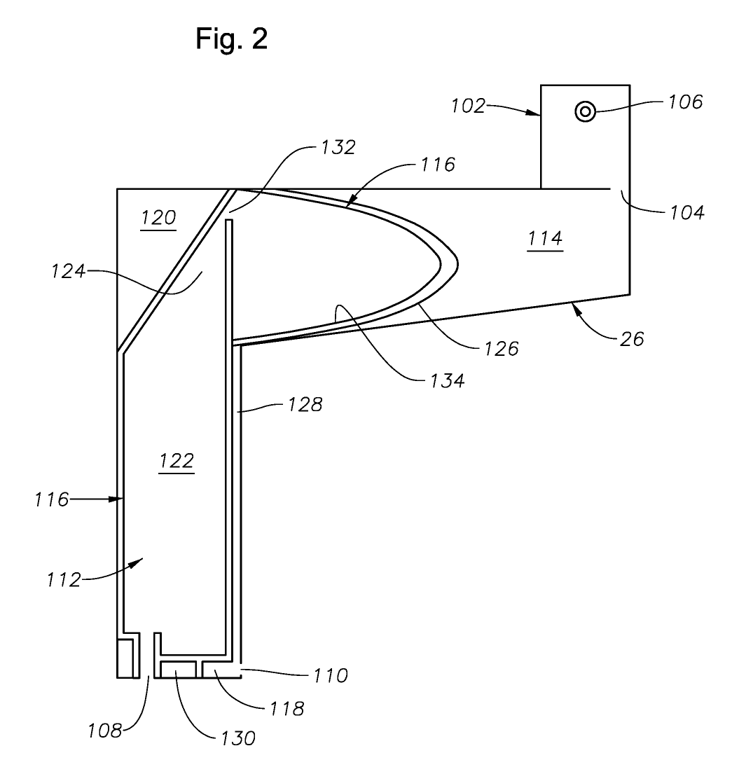

The preferred embodiments of the present invention and their advantages are best understood by referring to FIGS. 1-2 of the drawings, like numerals being used for like and corresponding parts of the various drawings.

As shown in FIG. 1, microsurgical system 10 includes a pressurized gas source 12, an isolation valve 14, a vacuum proportional valve 16, an optional second vacuum proportional valve 18, a pressure proportional valve 20, a vacuum generator 22, a pressure transducer 24, a surgical cassette 100 having an aspiration chamber 26, a fluid level sensor 28, a pump 30, a collection bag 32, an aspiration port 34, a surgical device 36, a computer or microprocessor 38, and a proportional control device 40. The various components of system 10 are fluidly coupled via fluid lines 44, 46, 48, 50, 52, 54, 56, and 58. The various components of system 10 are electrically coupled via interfaces 60, 62, 64, 66, 68, 70, 72, 74, and 76. Valve 14 is preferably an “on/off” solenoid valve. Valves 16-20 are preferably proportional solenoid valves. Vacuum generator 22 may be any suitable device for generating vacuum but is preferably a vacuum chip or a venturi chip that generates vacuum when isolation valve 14 and vacuum proportional valves 16 and/or 18 are open and gas from pressurized gas source 12 is passed through vacuum generator 22. Pressure transducer 24 may be any suitable device for directly or indirectly measuring pressure and vacuum. Fluid level sensor 28 may be any suitable device for measuring the level of a fluid 42 within aspiration chamber 26 but is preferably capable of measuring fluid levels in a continuous manner. Fluid level sensor 28 is most preferably an optical sensor capable of measuring fluid levels in a continuous manner. Pump 30 may be any suitable device for generating vacuum but is preferably a peristaltic pump, a scroll pump, or a vane pump. Microprocessor 38 is capable of implementing feedback control, and preferably PID control. Proportional controller 40 may be any suitable device for proportionally controlling system 10 and/or surgical device 36 but is preferably a foot controller.

System 10 preferably utilizes three distinct methods of controlling aspiration, vacuum control, suction control, and flow control. These methods are more fully described in co-pending U.S. application Ser. No. 11/158,238 and co-pending U.S. application Ser. No. 11/158,259, both of which are commonly owned with the subject application and are incorporated herein by reference.

In each of these methods, vacuum may be provided to surgical device 36 and aspiration chamber 26 via fluid lines 50, 56, and 58. Aspiration chamber 26 fills with fluid 42 aspirated by surgical device 36. Fluid 42 includes liquid infusion fluid as well as aspirated ophthalmic tissue.

As shown in FIGS. 1-2, a surgical cassette 100 preferably has an aspiration chamber 26 and an aspiration source chamber 102. Aspiration source chamber 102 preferably has a small volume relative to aspiration chamber 26. An entry opening 104 fluidly couples aspiration chamber 26 and aspiration source chamber 102. A port 106 fluidly couples aspiration source chamber 102 and fluid line 50. As discussed hereinabove, fluid line 50 is fluidly coupled to vacuum generator 22. Aspiration chamber 26 is comprised of sensing chamber 112 and overflow chamber 114. Sensing chamber 112 and overflow chamber 114 are fluidly coupled at an angle that is most preferably about 90 degrees. A liquid/gas separating structure 116 divides sensing chamber 112 into an anterior section 118 and a posterior section 120. Fluid level sensor 28 measures the fluid level in posterior section 120. An entry opening 108 fluidly couples anterior section 118 and fluid line 56. An entry opening 110 fluidly couples anterior section 118 and fluid line 52.

Liquid gas separating structure 116 preferably includes a hollow bore 122 terminating in a converging nozzle 124, a curved deflector 126 disposed in overflow chamber 114, a drain channel 128 disposed in sensing chamber 112 and fluidly coupled to overflow chamber 114 and anterior section 118, and an entry opening 130 fluidly coupling anterior section 118 and posterior section 120. Converging nozzle 124 has an exit opening 132 fluidly coupled to overflow chamber 114. Entry 108 preferably terminates within hollow bore 122 above entry 130. Deflector 126 preferably has a curved shape and is oriented such that opening 132 is located inside its concave surface, and opening 104 is located outside its convex surface. Deflector 126 most preferably has a generally parabolic shape. Deflector 126 has interior surface 134 that is preferably sharp. Deflector 126 is preferably sized so that the portion of posterior section 120 above converging nozzle 124 is fluidly coupled with opening 104 via overflow chamber 114.

Cassette 100 is preferably molded from a plastic material. Aspiration chamber 26 and liquid/gas separating structure 116 are preferably integrally molded into cassette 100. Alternatively, liquid/gas separating structure 116 may be separately molded from a plastic material and then frictionally secured and/or bonded within aspiration chamber 26. In either case, liquid/gas separating structure 116 is preferably opaque.

As shown best in FIG. 1, liquid 42 is present in aspiration chamber 26, and air 43 is present in aspiration chamber 26 above liquid 42. When the surgical system supplies vacuum to aspiration chamber 26, some liquid 42 is mixed with air 43, typically on or in air bubbles. Liquid infusion fluid and ophthalmic tissue from surgical device 36, which also may be a liquid/gas mixture, enters anterior section 118 via entry 108. Fluid level sensor 28 measures the liquid level in posterior section 120 of sensing chamber 112. Because entry 108 terminates above opening 130, the buoyancy of any bubbles present in the liquid/air mixture prevents the bubbles from passing through opening 130 into posterior section 120. By separating air bubbles into anterior section 118 of aspiration chamber 26, liquid/gas separating structure 116 allows fluid level sensor 28 to measure the level of liquid in aspiration chamber 26 in an accurate, reliable manner and eliminates any errors associated with air bubbles. The opaque nature of bubble separating structure 116 eliminates any errors of fluid level sensor 28 associated with ambient light entering into cassette 100.

As the liquid/air mixture travels into converging nozzle 124, the flow velocity increases. The increased velocity deforms the fluid films, separates bubbles and forces them to coalesce, and drives the liquid to the perimeter of the flow path. Some of the liquid then flows back down into anterior section 118, and does not contribute to bubble formation. This phenomenon makes it very difficult for any bubbles to form at opening 132. Those bubbles that do form at opening 132 are usually weak due to the limited supply of liquid from which to form a film. These bubbles are usually broken by the high velocity air emitted from opening 132.

During initial air operation of surgical device 36, the liquid flow rate into aspiration chamber 26 greatly increases. The resulting surge sends a stream of liquid out of opening 132 and into overflow chamber 114. The curved shape of deflector 126 directs this stream of liquid toward the bottom of overflow chamber 114 and away from opening 104 and port 106. In addition, the sharpened interior surface 134 of deflector 126 breaks any bubbles that form at opening 132 and do not immediately burst. Drain channel 128 drains liquid in overflow chamber 114 to the bottom of anterior section 118. Drain channel 128 ensures that the fluid level in anterior section 118 and posterior section 120 remains equal.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. A surgical cassette, comprising:
an aspiration source chamber;
an aspiration chamber, comprising:
an overflow chamber;
a sensing chamber;
a liquid/gas separating structure dividing said sensing chamber into an anterior section and a posterior section, said separating structure comprising:
a converging nozzle fluidly coupled to said anterior section;
a curved deflector disposed in said overflow chamber; and
a drain channel disposed in said sensing chamber and fluidly coupled to said overflow chamber and said anterior section;
a first opening to said anterior section for receiving a liquid/gas mixture from a surgical device;
an exit from said converging nozzle for directing said liquid/gas mixture toward a concave surface of said deflector; and
a second opening disposed outside a convex surface of said deflector and fluidly coupling said overflow chamber and said aspiration source chamber.

2. The surgical cassette of claim 1 wherein said first opening has a termination, and further comprising a third opening disposed below said termination of said first opening and fluidly coupling said anterior section and said posterior section.

3. The surgical cassette of claim 2 wherein said posterior section collects liquid for measuring a liquid level in said aspiration chamber.

4. The surgical cassette of claim 1 wherein said converging nozzle increases flow velocity of said liquid/gas mixture and impedes bubble formation.

5. The surgical cassette of claim 1 wherein said concave surface of said deflector is sharp.

6. The surgical cassette of claim 1 wherein said deflector breaks bubbles entering said overflow chamber.

7. The surgical cassette of claim 1 wherein said liquid/gas separating structure is opaque.
